(19) United States
(12) Patent Application Publication
(10) Pub. No.: US 2010/0298946 A1
(43) Pub. Date: Nov. 25, 2010

(54) ARTIFICIAL KNEE JOINT INCLUDING PLURAL FLEXIONS IN A FEMUR JOINT MEMBER

(75) Inventors: Doo-Hoon Sun, Seoul (KR); Yong-Sik Kim, Seoul (KR); Jung-Sung Kim, Chungcheongnam-do (KR); Byung-Soo Kim, Seoul (KR); Chang-Dong Han, Seoul (KR); Eun-Kyoo Song, Gwangju (KR); Ye-Yeon Won, Gyeonggi-do (KR)

Correspondence Address: Workman Nydegger;1000 Eagle Gate Tower, 60 East South Temple, Salt Lake City, UT, 84111, US

(73) Assignee: CORENTEC INC., Chungcheongnam-do (KR)

(21) Appl. No.: 12/811,691

(22) PCT Filed: Jan. 8, 2009
(86) PCT No.: PCT/KR2009/000100

§ 371 Date: Aug. 12, 2010

(30) Foreign Application Priority Data
Jan. 8, 2008 (KR) 10-2008-0002239

(51) Int. Cl. A61F 2/38

(52) U.S. Cl. 623/20.24

(57) ABSTRACT

An artificial knee joint in which plural flexions are formed at a femur joint member includes a femur joint member which is joined to an end portion of a tibia near a femur; a tibia joint member which is joined to an end portion of a femur near a tibia; and a bearing member between the femur joint member and the tibia joint member, wherein the femur joint member includes plural side flexions which have various radii of curvature in order to make the contact surface become enlarged so that the stress may be dispersed at the contact portion with the tibia.

### CROSS-REFERENCE TO RELATED APPLICATIONS

Not applicable.

### BACKGROUND OF THE INVENTION

1. Technical Field

The present invention relates generally to an artificial knee joint which can replace a natural knee joint. More specifically, this invention is directed to an artificial knee joint having a femur joint member which is attached to an end portion of the femur near the tibia, and a tibia joint member which is attached to an end portion of the tibia near the femur, wherein the artificial knee joint evenly disperses the stress of both the femur joint member and the tibia joint member regardless of the kind of knee movement by enlarging the contact surface between the femur joint member and the tibia joint member. Especially, the artificial knee joint classifies the curvatures of the femur and tibia joint members according to the degree of rotation so as to enlarge the contact area in any state, thus effectively dispersing the stress caused by the repeated load. The tibia joint member includes a bearing member near the contact portion with the femur joint member, thus forming the curvature of the bearing member corresponding to the curvature of the femur joint member.

2. Background Art

Among the many joints in the body, the knee joint is the junction between the tibia and the femur. Because of the wearing of the knee joint, the aging of the bone tissue and accidents, the number of patients for whom recovery is impossible is gradually increasing. The knee joint is the joint which is located between the lower end of the femur, the upper end of the tibia, and the back of the patella, and functions to bend the leg backwards at the knee.

The back of the patella is covered by cartilage 4 mm to 6 mm thick. The patella moves up and down along the articular surface which is in front of the end portion of a thighbone (the femur) while the knee is bent or stretched (at the patellofemoral joint), thus improving the knee stretching force of the musculus quadriceps femoris. Pressure acting on the patellofemoral joint when a person walks on a flat ground is equal to half of his or her weight. When a person goes up the stairs, a force which is 3 times as great as his or her weight acts on the patellofemoral joint. Further, when a person sitting in a squatting position stands up, a force which is 8 times as great as his or her weight acts on the patellofemoral joint. An articular capsule extends from the edge of the lower end of the femur to the edge of the upper end of the tibia. In addition to the above components, the inner and outer collateral ligaments, the cruciate ligaments of knee in the articular capsule, and other strong ligaments strengthen the connection of the bones and limit the moving direction and range of the bones.

The symptoms that appear when the meniscus of the knee joint is damaged will now be described. Here, the meniscus of the knee joint is the cartilaginous tissue which is located between the femur and the tibia forming the knee joint. The meniscus is positioned between the articular cartilage to absorb shocks acting on the knee joint, supply nutrients to the articular cartilage, provide the joint with stability, allow the knee joint to move smoothly, and transmit the load imposed by the weight.

Generally, the meniscus includes a medial meniscus and a lateral meniscus. As for Europeans and Americans, the medial meniscus is larger in size and smaller in mobility than the lateral meniscus, so that the medial meniscus is easily damaged. However, it is known the lateral meniscus of Koreans is more heavily damaged than is the medial meniscus.

The damage to the meniscus is one type of damage frequently occurring in knee joints. The meniscus is frequently damaged during athletic sports, mountain climbing or daily life. This easily occurs when the bent knee is rotated, that is, when torsional force is applied to the knee joint. When excessive external force acts on the knee joint, a cruciate ligament or collateral ligament may be damaged and the tibia may be fractured.

Most symptoms of disease taking place in the patella do not reveal any noteworthy external injury, and are caused by structural and functional disorders of the patellofemoral joint. When the leg is bent abnormally outward or the foot is bent excessively outward, excessive force is repeatedly exerted on the patellofemoral joint, thus leading to the malacia of the articular cartilage. Even when the knee joint is not used for a lengthy period of time, the musculus quadriceps femoris may become weak, thus causing damage to the patella.

When there are structural disorders of the patellofemoral joint, it is possible to wear an orthosis to stabilize the patella. Meanwhile, when the damage to the patellofemoral joint is severe, surgical procedures may be performed to replace the damaged joint with an artificial knee joint.

Recently, a surgical procedure for replacing a patient's joint which has been so severely damaged that it is impossible to recover with an artificial joint has been widely performed. Metal, ceramic or polyethylene is used for the motion part of the artificial joint, thus providing superior mechanical characteristics, reducing the coefficient of friction, and enhancing biocompatibility. Generally, the artificial knee joint includes a femur part, a tibia part, and a bearing part which is provided between the femur part and the tibia part and corresponds to cartilage. Here, the femur part and the tibia part are most commonly made of a metal alloy, while the cartilage part is made of polyethylene or the like. The tibia part is secured by an insertion part which is inserted into an end of the tibia near the knee joint. The insertion part is attached to the marrow of the tibia. However, if a load is repetitively applied to the knee joint, it is difficult for the artificial knee joint to achieve a sufficient effect, because of its structural defects. The femur part and the tibia part may be damaged by the continuous imposition of a load. Especially should the bearing part be broken, big problems occur.

### Technical Problem

Accordingly, the present invention has been made keeping in mind the above problems occurring in the prior art, and an object of the present invention is to provide an artificial knee joint including a plurality of flexions in a femur joint member, in which the curvatures of the femur joint member and a bearing member are adjusted to evenly distribute the stress, thus imparting a contact portion when the knee moves with a larger contact area, therefore dispersing the stress.

Another object of the present invention is to provide an artificial knee joint including a plurality of flexions in a femur joint member, in which the femur joint member and a bearing member have corresponding curvature when seen from the front, because horizontal rotation may occur at the contact portion of a femur and a tibia, and the femur joint member and the bearing member have circular curvature so as to allow knee movement to be more smoothly and easily performed, thus preventing the concentration of stress and dispersing the stress when leaning to one side.

A further object of the present invention is to provide an artificial knee joint including a plurality of flexions in a femur joint member, in which the femur joint member has various curvatures when seen from the side, thus allowing the knee to be smoothly rotated and bent, enabling the knee to be bent at a larger angle, and preventing the femur from becoming separated from the tibia.

Yet another object of the present invention is to provide an artificial knee joint including a plurality of flexions in a femur joint member, which enlarges a contact area between the upper portion of a bearing member and a lower portion of the femur joint member, thus evenly dispersing stress caused by load acting in a direction from an upper position to a lower position, therefore improving the durability of the femur joint member, the bearing member, and the tibia joint member, and providing more stable movement to a patient who underwent a surgical procedure.

Still another object of the present invention is to provide an artificial knee joint including a plurality of flexions in a femur joint member, which adjusts the curvature of the side of the lower portion of the femur joint member, thus allowing the knee to be more naturally bent forward or backward, permitting the load to be evenly transmitted, and dispersing stress.

Another object of the present invention is to provide an artificial knee joint including a plurality of flexions in a femur joint member, which adjusts the curvature of the front of the lower portion of the femur joint member, thus dispersing stress at the site where the load is concentrated, because one side of the femur joint member is lifted up and a large load acts on only one side when the knee is slightly rotated leftward or rightward.

### Technical Solution

In order to accomplish the above objects, the present invention provides an artificial knee joint including flexions in a femur joint member, having a femur joint member attached to an end portion of a femur, a tibia joint member attached to an end portion of a tibia, and a bearing member positioned between the femur joint member and the tibia joint member, wherein the femur joint member comprises a contact portion which is in contact with the bearing member so as to disperse stress, and the contact portion comprises side flexions having various curvature radii so that a contact surface between the femur joint member and the bearing member is large when viewed from a side.

According to an aspect of the invention, the side flexions may include a first side flexion, a second side flexion and a third side flexion, the second side flexion being connected to the third side flexion, the first side flexion being connected to the second side flexion, curvature radii being reduced from the third side flexion to the first side flexion.

In another aspect of the invention, the contact portion of the femur joint member may further include a front flexion having a gently curved surface so as to provide a large contact area between the contact portion of the femur joint member and the bearing member, when viewed from a front, thus dispersing stress.

In yet another aspect of the invention, the bearing member may include a depressed portion having curvature which substantially corresponds to curvature of the side flexions of the contact portion of the femur joint member, thus increasing the contact area and dispersing stress.

In still another aspect of the invention, the bearing member may include a concave portion having curvature which substantially corresponds to curvature of the front flexion of the contact portion of the femur joint member, thus increasing the contact area and dispersing stress.

In yet another aspect of the invention, the bearing member may further include a front projecting part which is projected from a front portion of the bearing member to a predetermined height, and a rear projecting part which is projected from a rear portion of the bearing member to a predetermined height, thus preventing the femur joint member from being dislocated from the bearing member.

In still another aspect of the invention, the artificial knee joint may include a contact portion making contact with a bearing member so as to disperse stress, the contact portion including side flexions having various curvature radii so that a contact surface between the femur joint member and the bearing member is large when viewed from a side, the side flexions including a first side flexion, a second side flexion and a third side flexion, the second side flexion being connected to the third side flexion, the first side flexion being connected to the second side flexion, curvature radii being reduced from the third side flexion to the first side flexion.

In yet another aspect of the invention, the artificial knee joint may further include a front flexion having a gently curved surface so as to provide a large contact area between the contact portion of the femur joint member and the bearing member, when viewed from a front, thus dispersing stress.

### ADVANTAGEOUS EFFECTS

As described above, the present invention can accomplish the following effects by the above-mentioned technical solutions and the construction and operation to be mentioned later.

According to the present invention, it is advantageous in that the curvatures of the femur joint member and a bearing member are adjusted to evenly distribute the stress, thus imparting a contact portion when the knee moves with a larger contact area, therefore dispersing the stress.

The present invention is advantageous in that the femur joint member has various curvatures when seen from the side, thus allowing the knee to be smoothly rotated and bent, enabling the knee to be bent at a larger angle, and preventing the femur from becoming separated from the tibia.

The present invention is advantageous in that the femur joint member and a bearing member have corresponding curvature when seen from the front, because horizontal rotation may occur at the contact portion of a femur and a tibia, and the femur joint member and the bearing member have circular curvature so as to allow knee movement to be more smoothly and easily performed, thus preventing the concentration of stress and dispersing the stress when leaning to one side.

The present invention is advantageous in that a contact area between the upper portion of a bearing member and a lower portion of the femur joint member is enlarged, thus evenly dispersing stress caused by load acting in a direction from an upper position to a lower position, therefore improving the durability of the femur joint member, the bearing member, and the tibia joint member, and providing more stable movement to a patient who underwent a surgical procedure.

The present invention is advantageous in that the curvature of the lower portion of a femur joint member is adjusted, so that a contact area with the upper portion of a tibia joint member or a bearing member is enlarged, thus dispersing stress, therefore preventing an artificial knee joint from being broken by a load, and increasing the durability and life-span of the artificial knee joint.

The present invention is advantageous in that the curvature of the lower portion of a femur joint member is adjusted, thus dispersing stress concentrated when the knee is greatly rotated and the load is thereby transmitted in various directions, therefore reducing the wear caused by friction, and realizing a sturdy artificial knee joint.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is a view illustrating the knee to which a conventional artificial knee joint is implanted;

FIG. 2 is a view illustrating a femur joint member of an artificial knee joint according to an embodiment of the present invention;

FIG. 3 is a view illustrating a first side flexion in the femur joint member according to the present invention;

FIG. 4 is a view illustrating a second side flexion in the femur joint member according to the present invention;

FIG. 5 is a view illustrating an artificial knee joint according to an embodiment of the present invention;

FIG. 6 is a view illustrating the artificial knee joint according to the embodiment of the present invention;

FIG. 7 is a view illustrating a front flexion in the femur joint member according to the present invention;

FIG. 8 is a view illustrating an artificial knee joint according to another embodiment of the present invention;

FIG. 9 is a view illustrating the concentration of stress in a contact portion of the conventional artificial knee joint;

FIG. 10 is a view illustrating the dispersion of stress because of the enlargement of the contact area according to the present invention;

FIG. 11 is a sectional view illustrating the contact area between the femur joint member and a bearing member according to the present invention;

FIG. 12 is a graph illustrating relation between the contact area and the dispersion of stress, in which the stress dispersion effect achieved by the enlargement of the contact area according to the present invention is represented; and

FIG. 13 is a table illustrating the effect achieved by the dispersion of stress and reduction in the load according to the present invention.

### DETAILED DESCRIPTION OF THE PREFERRED EMBODIMENTS

### Best Mode

Hereinafter, an artificial knee joint including a plurality of flexions in a femur joint member according to the preferred embodiment of the present invention will be described in detail with reference to the accompanying drawings.

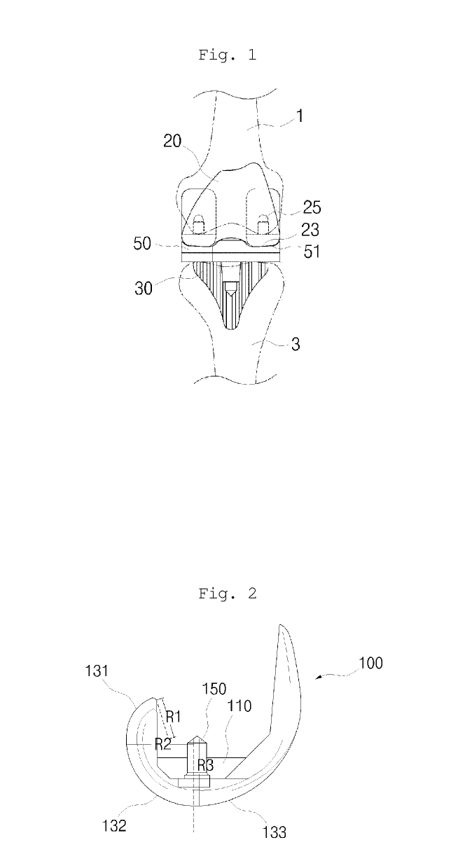

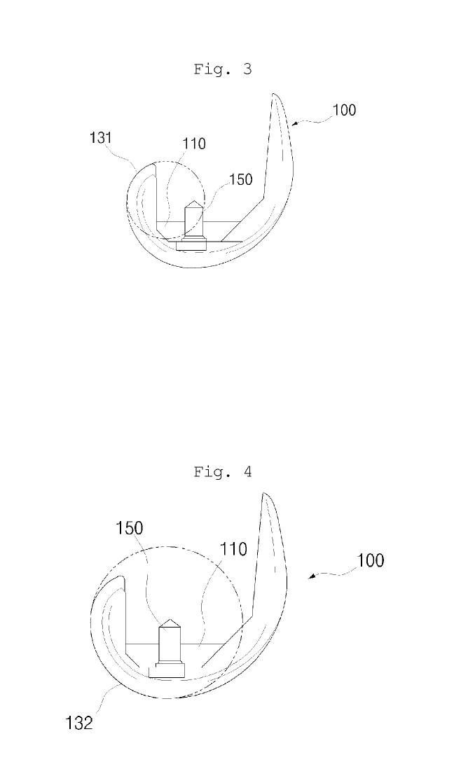

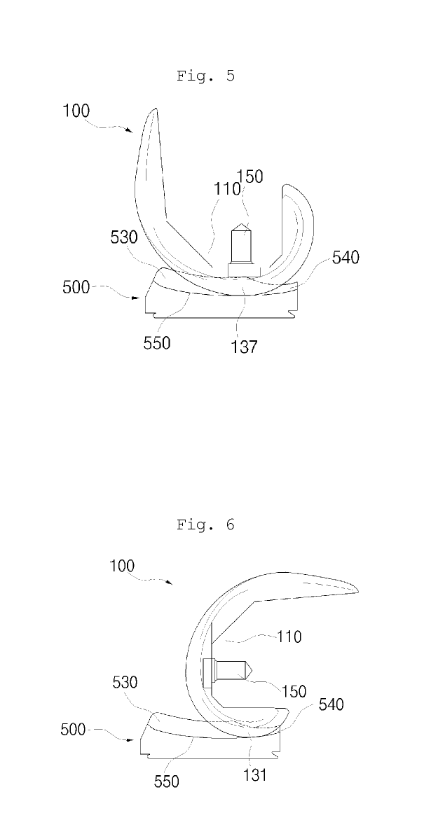

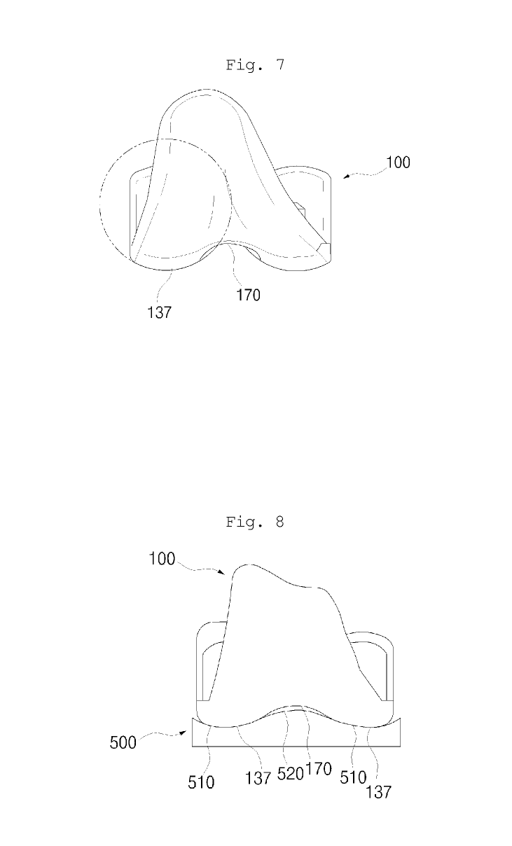

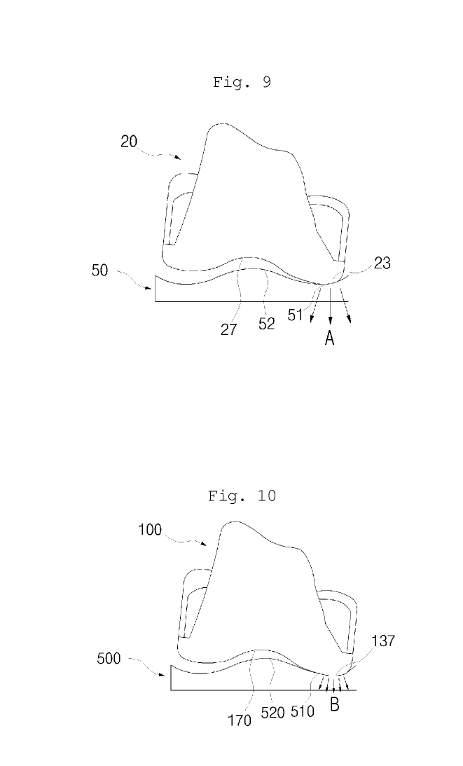

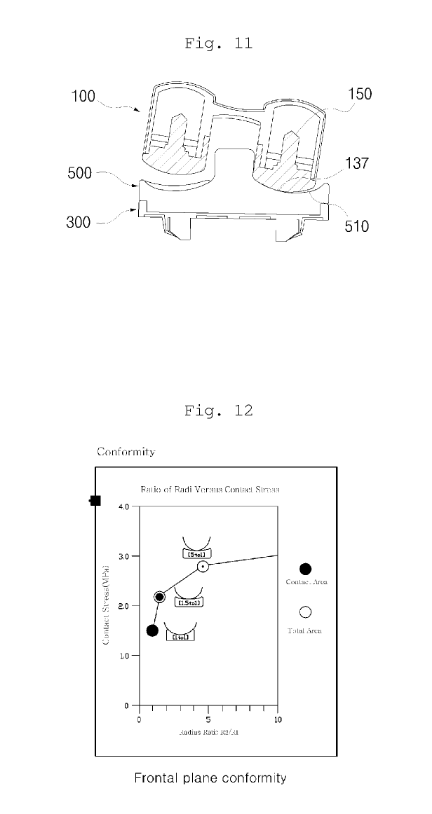

FIG. 2 is a view illustrating a femur joint member of an artificial knee joint according to an embodiment of the present invention, FIG. 3 is a view illustrating a first side flexion in the femur joint member according to the present invention, FIG. 4 is a view illustrating a second side flexion in the femur joint member according to the present invention, FIG. 5 is a view illustrating an artificial knee joint according to an embodiment of the present invention, FIG. 6 is a view illustrating the artificial knee joint according to the embodiment of the present invention, FIG. 7 is a view illustrating a front flexion in the femur joint member according to the present invention, FIG. 8 is a view illustrating an artificial knee joint according to another embodiment of the present invention, FIG. 9 is a view illustrating the concentration of stress in a contact portion of the conventional artificial knee joint, FIG. 10 is a view illustrating the dispersion of stress because of the enlargement of the contact area according to the present invention, and FIG. 11 is a sectional view illustrating the contact area between the femur joint member and a bearing member according to the present invention.

Referring to FIGS. 2 to 11, the artificial knee joint of the present invention includes a femur joint member 100 which is attached to the lower portion of the femur 1 of FIG. 1, a tibia joint member 300 which is attached to the upper portion of the tibia 3, and a bearing member 500 which is provided between the femur joint member and the tibia joint member and serves as cartilage. The femur joint member 100 is in friction contact with the bearing member 500, and the bearing member is subjected to stress generated by load which is transmitted from the upper portion of the femur joint member. Further, the femur joint member is in contact with the bearing member, so that the tibia may move forward, backward, rightward and leftward when the leg is moved by the ligaments. Thus, it is preferable that the femur joint member and the bearing member be in contact with each other in various manners according to the curvature when the knee joint moves. This serves to properly disperse the stress.

The femur joint member 100 has a “U” shape and is made of a biocompatible material. The upper portion of the femur joint member 100 has a part for receiving the femur 1, and the lower portion thereof has a curved surface which has various curvatures and approximates a spherical shape. The femur joint member 100 includes a femur receiving part 110 and a locking protrusion 150. Part of the femur is cut, so that it is received by the femur receiving part of the femur joint member 100. The locking protrusion 150 couples the femur more firmly to the femur receiving part 110. Further, the femur joint member 100 has on its lower surface curved contact portions which are in contact with the bearing member 500 that will be described below, and a depressed portion 170 which is concavely formed between the contact portions.

The femur receiving part 110 is positioned at the interior of the upper portion of the femur joint member 100, and is firmly coupled to the incision surface of the lower portion of the femur. Preferably, the femur receiving part 110 has a rough surface or is made of a porous material so as to be firmly coupled to the femur.

The locking protrusion 150 extends upwards from the femur receiving part 110 to be inserted into the femur 1, and has the shape of a screw. More preferably, the locking protrusion 150 has a locking part which is shaped to be inserted into the femur and firmly grip the bone tissue of the femur.

The contact portions are the important part of the present invention. When the femur moves while making contact with concave portions 510 of the bearing member 500, the contact portions are preferably shaped to maximally enlarge the contact area, thus naturally dispersing stress. Here, the curvature of the contact portion when seen from the side is different from that of the contact portion when seen from the front. The different curvatures enable natural movement even when the knee joint moves forward and backward, and increases the contact area, thus dispersing stress. Further, when the knee joint is slightly abducted leftward or rightward or one contact portion is slightly lifted, a large stress is concentrated on the other contact portion. In this case, the different curvatures increase the contact area so as to disperse stress. When seen from the side, the contact portion includes a first side flexion 131, a second side flexion 132, and a third side flexion 133. Meanwhile, when seen from the front, the contact portion includes a front flexion 137.

The side flexion will be described with reference to FIGS. 2 to 6. First, as shown in FIG. 2, the side flexion includes the first side flexion 131, the second side flexion 132 and the third side flexion 133. When the knee is actually bent, so that the femur joint member 100 rolls around an axis, the knee can be more reliably bent owing to the curvature radii which are gradually reduced, and thus can be bent at a larger angle (it is referred to as roll back, which allows the knee to be bent at an angle larger than an angle at which the knee is generally bent). As shown in FIGS. 2 and 3, the first side flexion 131 has the smallest curvature radius, that is, R1. The second side flexion 132 has the curvature radius of R2 which is larger than R1, as shown in FIGS. 2 and 3, and the third side flexion 133 has the largest curvature radius, that is, R3, as shown in FIG. 2. As such, respective portions have different curvature radii. Thus, as shown in FIGS. 5 and 6, in the state in which the knee is not bent at normal times, the second and third side flexions 132 and 133 come into contact with the concave portion 510 of the bearing member 500, thus providing a larger contact surface, therefore dispersing stress. Meanwhile, when the knee is bent, as shown in FIG. 6, the first and second side flexions 131 and 132 come into contact with the concave portion. Consequently, when the knee rotates, the large contact area is continuously maintained to disperse stress. Moreover, the curvature radii R1, R2, and R3 are gradually reduced from the third flexion 133 to the first flexion 131, thus enabling the roll back of the knee when the knee is bent, and more effectively preventing the femur from becoming dislocated from the tibia even if the knee is bent at a large angle.

Next, the front flexion 137 will be described with reference to FIGS. 7 to 11. The front flexion 137 is referred to as the flexion of the contact portion when the femur joint member 100 is seen from the front. When the front flexion 137 is in contact with the concave portion 510 of the bearing member 500, they are in contact with each other in a transverse direction from left to right in a gentle curve. Preferably, the gentle curve is formed to have a predetermined curvature radius as shown in FIG. 7. Generally, the knee of the human body may move slightly leftward or rightward. Normally, as shown in FIG. 8, the left and right contact portions of the femur joint member 100 are in contact with the concave portions 510 of the bearing member 500. However, when the knee is slightly abducted leftward or rightward, as shown in FIGS. 9 and 10, one contact portion is slightly lifted, and the other contact portion makes closer contact with the concave portion, so that the stress may be concentrated on one side. Thus, in the conventional artificial knee joint shown in FIG. 9, the contact portion positioned at the side on which the stress is concentrated is narrow, so that the concentrated distribution A of stress is obtained because of the narrow contact area. However, as shown in FIG. 10, although one contact portion is lifted and the other contact portion makes closer contact with the bearing member, stress can be dispersed as shown by the arrows B owing to the large contact area, thus solving the concentration of stress founded in the prior art to some extent. The enlargement of the contact portion by the formation of the front flexion 137 can achieve the dispersion of stress. This is more clearly shown in FIG. 11 which is a sectional view illustrating the contact of the femur joint member 100 and the bearing member 500. Hence, it is preferable that the curvature of the front flexion 137 correspond to the curvature of the bearing member 500.

The tibia joint member 300 is fitted into and secured to the upper portion of the tibia in the typical artificial knee joint, is made of a biocompatible material, and supports the bearing member 500 which will be described below. Further, the tibia joint member may be formed in various types, and is typically classified into a mobile type and a fixed type according to the coupling relation between the tibia joint member and the bearing member. In the case of the mobile type tibia joint member, the bearing member 500 may rotate or move forward and backward on the tibia joint member 300, thus achieving the more natural movement of the knee joint. However, in the case of the fixed type tibia joint member, the bearing member 500 is fixed to the tibia joint member 300 so as not to be moved. The fixed type tibia joint member makes free movement difficult, but realizes stable movement owing to firm coupling. Thus, in the fixed type tibia joint member, the bearing member 500 may be integrated with the tibia joint member 300 into a single structure. In this case, it is natural that the effect realized by the bearing member 500 which is coupled to and makes contact with the femur joint member 100 be also achieved by the tibia joint member 300. Thus, the effect achieved by the components and operation of the bearing member 500 according to the present invention can be achieved by the tibia joint member 300. This falls within the scope of the present invention.

The bearing member 500 is provided between the femur joint member 100 and the tibia joint member 300 to serve as the cartilage of the human body. Thus, the bearing member 500 is preferably made of polyethylene unlike the material of the femur joint member and the tibia joint member. Polyethylene does not produce impurities when it wears, and does not generate heat due to friction, so that it withstands friction well, and has a smooth surface to permit natural frictional contact. The bearing member 500 includes the concave portions 510 which are provided on the upper portion of the bearing member and make contact with the contact portions of the femur joint member 100 when seen from the front. A convex portion 520 is provided between the concave portions 510. Front and rear projecting parts 530 and 540 are provided on opposite ends of the bearing member 500 when seen from the side. A depressed portion 550 is further included.

The concave portions 510 are portions which are provided on both sides of the artificial knee joint according to the present invention when viewing the bearing member 500 from the front. Friction occurs between the concave portions and the contact portions, when the concave portions are in contact with the contact portions of the femur joint member 100, so that the femur joint member 100 rotates. Thus, since the contact concentrates the load on the concave portions, it is necessary to maximize the contact area so as to prevent the concentration of stress. Therefore, the concave portion 510 preferably has curvature corresponding to the front flexion 137 of the femur joint member 100. Thereby, as described above, when the concave portions are slightly lifted leftwards or rightwards at the front flexions 137, contact occurs on only one side, so that the stress is concentrated on the contact side. However, owing to the increase in the contact area, the stress can be dispersed.

The convex portion 520 protrudes upwards between the concave portions 510 when viewing the bearing member 500 from the front, and corresponds to the depressed portion 170 of the femur joint member 100. However, it is not a contact portion but receives the patella which allows the knee to bend. Since the mechanism for bending the knee because of the patella and the ligaments is well known by those skilled in the art, a detailed description will be omitted from herein.

The front and rear projecting parts 530 and 540 are projected from the front and rear portions, when viewing the bearing member 500 from the side, to predetermined heights. Especially the front projecting part 530 prevents the femur joint member 100 from becoming separated from the bearing member 500 when a patient having the artificial knee joint of the present invention bends his or her knee at a large angle. The rear projecting part 540 prevents dislocation when the knee is bent at a large angle and rolled back, thus guaranteeing the stable movement of the knee joint.

When viewing the bearing member 500 from the side, the depressed portion 550 is the portion which is in contact with the first side flexion 131, the second side flexion 132 or the third side flexion 133 at the contact portion of the femur joint member 100. Consequently, it is preferable that the depressed portion have curvature corresponding to that of the side flexions of the femur joint member as described above. Thereby, more stable coupling is achieved, and a sufficient contact area is acquired, thus effectively dispersing the stress.

The operation and use of the artificial knee joint according to the preferred embodiment of the present invention will be described below in detail with reference to the accompanying drawings.

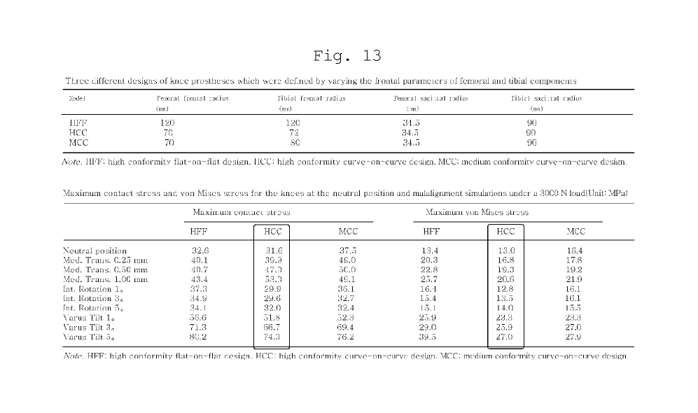

FIG. 5 is a view illustrating an artificial knee joint according to an embodiment of the present invention, FIG. 6 is a view illustrating the artificial knee joint according to the embodiment of the present invention, FIG. 8 is a view illustrating an artificial knee joint according to another embodiment of the present invention, FIG. 9 is a view illustrating the concentration of stress in a contact portion of the conventional artificial knee joint, FIG. 10 is a view illustrating the dispersion of stress because of the enlargement of the contact area according to the present invention, FIG. 11 is a sectional view illustrating the contact area between the femur joint member and a bearing member according to the present invention, FIG. 12 is a graph illustrating relation between the contact area and the dispersion of stress, in which the stress dispersion effect achieved by the enlargement of the contact area according to the present invention is represented, and FIG. 13 is a table illustrating the effect achieved by the dispersion of stress and reduction in the load according to the present invention.

The operation of the artificial knee joint according to the present invention will be described with reference to FIGS. 5 and 6. This achieves stable movement and the dispersion of stress in the case of the movement of the knee joint as seen from the side. Thereby, according to the present invention, the contact portion of the femur joint member 100 includes side flexions having various curvature radii. Preferably, the first side flexion 131, the second side flexion 132 and the third side flexion 133 may be included. First, in the state of FIG. 5 wherein the knee is not bent, the second side flexion 132 and the third side flexion 133 are in contact with the depressed portion 550 of the bearing member 500. Here, the second side flexion 132 and the third side flexion 133 must have curvature corresponding to that of the depressed portion 550 to make contact with the depressed portion over a relatively large area. Further, as shown in FIG. 6, even when the knee is bent, the depressed portion is also in contact with the second side flexion 132 and the first side flexion 131 which have different curvature radii, so that the femur joint member is in contact with the bearing member 500 or the tibia joint member 300 over a large area. Thereby, the stress is evenly distributed owing to the enlarged contact area. Further, since the curvature radii are sequentially reduced from the third side flexion to the first side flexion, the bending angle of the knee may become large although the degree the knee bends is small. This enables the roll back, thus achieving the natural movement of the knee joint. Consequently, the contact area with the bearing member 500 or the tibia joint member 300 is enlarged, thus preventing the concentration of stress, therefore preventing the artificial knee joint from being broken, improving durability, and increasing its life-span, in addition to making sure that the knee joint is stable when moving owing to the sequential difference in the curvature radius.

The operation of the artificial knee joint according to the present invention will be described with reference to FIGS. 8 to 11. The natural knee joint may slightly move leftward and rightward from the front, so that one contact portion may be lifted up. In consideration of such a movement of the natural knee joint, the present invention can compensate for stress concentrated on only one contact portion. When the knee into which the artificial knee joint of the present invention is implanted is not moved leftward or rightward as shown in FIG. 8, both contact portions are in contact with the concave portions 510 of the bearing member, so that the contact portions are subjected to dispersed stress. However, when the knee joint is abducted to one side, as shown in FIGS. 9 and 10, one side is lifted up and the stress is concentrated on the other side. In the conventional artificial knee joint of FIG. 9, the contact portion of the femur joint member 100 does not include a flexion, so that the contact area is very small and thus the load is concentrated on the small contact area. Thus, stress A concentrated by the load causes damage to the tibia joint member 300 as well as the bearing member 500, located at a lower position. However, in the artificial knee joint according to the present invention as shown in FIG. 10, even if contact occurs only in one contact portion and load is concentrated on the one contact portion, stress B may be relatively evenly dispersed owing to the large contact area. This is clearly shown in FIG. 11 which is the sectional view illustrating the case where a load is concentrated on one side of the artificial knee joint of the present invention.

The stress dispersion effect using the side and front flexions of the contact portion included in the femur joint member 100 can be clearly understood through FIGS. 12 and 13. The stress dispersion effect of the artificial knee joint according to the present invention will be described with reference to FIGS. 12 and 13. First, referring to FIG. 12 that shows the stress distribution as a function of the contact area, when the contact area is formed over a total area [1 to 1], stress (contact stress) is about 1.5 and relatively low. This is equivalent to the case where the ratio (R2/R1) of curvature radius is 1:1, and achieves the highest stress dispersion effect. However, when the ratio (R2/R1) of curvature radius is 1.5:1, the dispersion of stress is about 2.2 and is larger than the above-mentioned case. Meanwhile, when the ratio (R2/R1) of curvature radius is 5:1, the degree to which stress is concentrated approaches 3.0. As a result, as the contact area increases, stress is not concentrated but is effectively dispersed. Therefore, in the artificial knee joint of the present invention, the side and front flexions are provided on the contact portion of the femur joint member 100, thus providing a sufficiently large contact area, therefore effectively dispersing stress. FIG. 13 shows the numerical values for the stress distribution when contact portions are flat surfaces (HFF: high conformity flat-on-flat design), or are curved surfaces (HCC: high conformity curve-on-curve design). The left side of the lower table of FIG. 13 shows the experimental values of the maximum contact stress, in which the contact stress of HCC is smaller than that of HFF. Therefore, it can be seen that the dispersion effect of the stress is maximized when both the contact portion of the femur joint member 100 and the depressed portion of the bearing member 500 comprise curved surfaces, in the artificial knee joint of the present invention. Thus, according to the present invention, the side and front flexions of the contact portion of the femur joint member 100 are formed as curved surfaces, and the depressed portion of the bearing member 500 which is in contact with the femur joint member are formed as a curved surface to correspond to the curved surfaces of the side and front flexions, thus providing a means for effectively dispersing stress.

As such, according to the present invention, the contact portion of the femur joint member 100 includes side and front flexions, and the depressed portion of the bearing member 500 which is in contact with the femur joint member is formed as the curved surface, thus effectively dispersing stress, therefore preventing damage to the femur joint member, the bearing member and the tibia joint member, improving durability, and prolonging its life-span. Consequently, a sturdy artificial knee joint is realized.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. An artificial knee joint including flexions in a femur joint member, comprising:
a femur joint member attached to an end portion of a femur, a tibia joint member attached to an end portion of a tibia, and a bearing member positioned between the femur joint member and the tibia joint member,
wherein the femur joint member comprises a contact portion which is in contact with the bearing member so as to disperse stress,
the contact portion comprises side flexions having various curvature radii so that a contact surface between the femur joint member and the bearing member is large when viewed from a side, and a front flexion having a gently curved surface so as to provide a large contact area between the contact portion of the femur joint member and the bearing member, when viewed from a front, thus dispersing stress, and
the side flexions comprise a first side flexion, a second side flexion and a third side flexion, the second side flexion being connected to the third side flexion, the first side flexion being connected to the second side flexion, curvature radii being reduced from the third side flexion to the first side flexion.

2. (canceled)

3. (canceled)

4. The artificial knee joint according to claim 1, wherein the bearing member comprises a depressed portion having curvature which substantially corresponds to curvature of the side flexions of the contact portion of the femur joint member, thus increasing the contact area and dispersing stress.

5. The artificial knee joint according to claim 4, wherein the bearing member comprises a concave portion having curvature which substantially corresponds to curvature of the front flexion of the contact portion of the femur joint member, thus increasing the contact area and dispersing stress.

6. The artificial knee joint according to claim 4, wherein the bearing member further comprises a front projecting part which is projected from a front portion of the bearing member to a predetermined height, and a rear projecting part which is projected from a rear portion of the bearing member to a predetermined height, thus preventing the femur joint member from being dislocated from the bearing member.

7. A femur joint member, comprising:
a contact portion making contact with a bearing member so as to disperse stress, the contact portion comprising side flexions having various curvature radii so that a contact surface between the femur joint member and the bearing member is large when viewed from a side, and a front flexion having a gently curved surface so as to provide a large contact area between the contact portion of the femur joint member and the bearing member, when viewed from a front, thus dispersing stress, and
the side flexions comprising a first side flexion, a second side flexion and a third side flexion, the second side flexion being connected to the third side flexion, the first side flexion being connected to the second side flexion, curvature radii being reduced from the third side flexion to the first side flexion.

8. (canceled)

9. The artificial knee joint according to claim 5, wherein the bearing member further comprises a front projecting part which is projected from a front portion of the bearing member to a predetermined height, and a rear projecting part which is projected from a rear portion of the bearing member to a predetermined height, thus preventing the femur joint member from being dislocated from the bearing member.
